(19) United States
(12) Patent Application Publication
(10) Pub. No.: US 2024/0272177 A1
(43) Pub. Date: Aug. 15, 2024

(54) sFRP4 AS BLOOD BIOMARKER FOR THE NON-INVASIVE DIAGNOSIS OF ADENOMYOSIS

(71) Applicant: Roche Diagnostics Operations, Inc., Indianapolis, IN (US)

(72) Inventors: Manuel Dietrich, Brühl (DE); Felix Gruenewald, Penzberg (DE); Martin Hund, Horw (CH); Peter Kastner, Penzberg (DE); Martin Klammer, Muenchen (DE); Ruediger Laubender, Pullach im Isartal (DE); Heike Wegmeyer, München (DE); Ursula-Henrike Wienhues-Thelen, Krailling (DE)

(21) Appl. No.: 18/561,621

(22) PCT Filed: May 16, 2022
(86) PCT No.: PCT/EP2022/063114

§ 371 Date: Nov. 16, 2023

(30) Foreign Application Priority Data
May 17, 2021 (EP) 21174118.6

(51) Int. Cl. G01N 33/68; G01N 33/536; G01N 33/58; G16H 50/30

(52) U.S. Cl. CPC G01N 33/6893; G01N 33/536; G01N 33/58; G01N 33/6854; G16H 50/30; G01N 2333/4703; G01N 2800/361; G01N 2800/52

(57) ABSTRACT

The present invention relates to methods of assessing whether a patient has adenomyosis or is at risk of developing adenomyosis, to methods of selecting a patient for therapy of adenomyosis, and methods of monitoring a patient suffering from adenomyosis or being treated for adenomyosis, by determining the amount or concentration of sFRP4 in a sample of the patient, and comparing the determined amount or concentration to a reference.

The present invention relates to methods of assessing whether a patient has adenomyosis or is at risk of developing adenomyosis, to methods of selecting a patient for therapy of adenomyosis, and methods of monitoring a patient suffering from adenomyosis or being treated for adenomyosis, by determining the amount or concentration of sFRP4 in a sample of the patient, and comparing the determined amount or concentration to a reference.

### BACKGROUND

Adenomyosis is a heterogenous gynecologic condition. Patients with adenomyosis can have a range of clinical presentations. The most common presentation of adenomyosis is abnormal menstrual bleeding and dysmenorrhea. However, there could be also different forms of pain, nausea, or difficulties with urination. Patients with adenomyosis can also be asymptomatic and only coming to a clinician's attention during evaluation for infertility.

Adenomyosis is also associated with infertility and diagnosed in about 20% of infertile women undergoing assisted reproductive technologies (ART). (Puente J M et al. Reproductive Biology and Endocrinology, 2016; 14:60. Women with adenomyosis often have other associated gynecologic conditions such as endometriosis or leiomyomas, thus making the diagnosis and evaluating response to treatment challenging. (Pontis A et al. Gynecol Endocrinol, 2016; 32(9): 696-700). Currently, there are no standard diagnostic imaging criteria, and choosing the optimal treatment for patients is challenging.

Adenomyosis is defined as infiltration of benign endometrial glands and stroma into the myometrium, the outer muscle layer of the uterus. In contrast, Endometriosis is defined as a disease characterized by the presence of endometrium-like epithelium and/or stroma outside the endometrium and myometrium. There are three distinct adenomyosis sybtypes, depending on the morphology and location: internal adenomyosis, external adenomyosis and adenomyomas. Internal adenomyosis can be further classified into focal, diffuse and superficial adenomyosis (Bazot M et al. Fertil Steril. 2018; 109:389-397). Adenomyosis can also be classified based on Magnetic Resonance Imaging (MRI) findings, according to whether it is located in the outer or the inner uterine layer, into 4 subtypes I-IV. Common signs and symptoms of adenomyosis include heavy bleeding during menstruation (menorrhagia) and in-between menstrual periods (metrorrhagia), dysmenorrhea, chronic pelvic pain, dyspareunia and infertility, which seriously affects the quality of life of female patients. Furthermore Adenomyosis has a significant impact on fertility and in-vitro fertilization (IVF) outcomes (Chapron C et al. Hum Reprod Update. 2020; 26:392-411).

The prevalence of adenomyosis varies widely, with an average rate of 20%-25%. Approximately 20% of adenomyosis cases involve women of reproductive age (<40 years), with the remaining 80% occurring in women of late reproductive age (40-50 years). (Pontis A et al. Gynecol Endocrinol, 2016; 32(9): 696-700).

For women with bothersome symptoms who have completed childbearing, the removal of the uterus (hysterectomy) is the treatment of choice and the only definitive cure for adenomyosis. Uterus-preserving surgical excision of adenomyotic lesions or cysts is done in rare cases in patients with localized adenomyosis o cystic focal adenomyosis, however not a standard treatment. Uterine Artery Embolization (UAE) is a new treatment approach particularly in patients who are resistant to conventional therapy and those who wish to preserve the uterus. However, recurrence of adenomyosis signs and symptoms have been observed in more than 40% of patients (J. Zhou et al. PLOS One 2016; 1-15)

No specific drug is currently available to treat adenomyosis and there are no specific guidelines to follow for the best management.

The intended outcome of treating symptomatic adenomyosis is the relief of signs and symptoms, maintenance or improvement of fertility, while minimizing side effects.

Several non-hormonal (i.e., non-steroidal anti-inflammatory drugs (NSAIDs) and hormonal treatments (i.e., progestins, oral contraceptives, gonadotropin-releasing hormone [GnRH] analogues) are used to control pain symptoms and abnormal uterus bleeding in adenomyosis (S. Vannuccini et al. Fertility and Sterility® Vol. 109, No. 3, March 2018; A. Pontis et al. Gynecol Endocrinol. 2016; 3590: 1-5)

The diagnosis of adenomyosis is currently based on transvaginal ultrasonography (TVUS) and magnetic resonance imaging (MRI) with an overall sensitivity/specificity of 83.8%, 63.9% and 77.3%, 89.8% respectively. (Chapron C et al. Hum Reprod Update. 2020; 26:392-411). However currently, there are no standard diagnostic imaging criteria, and choosing the optimal treatment for patients is challenging. Both TVUS and transabdominal sonography characterize adenomyosis by identifying myometrial cysts, myometrial anteroposterior asymmetry and poorly defined foci of an abnormal myometrial echotexture. Findings on MRI include a large asymmetric uterus without leiomyomas, thickening of the junctional zone or an abnormal ratio of junctional zone to myometrial thickness. The junctional zone is the innermost myometrial layer.

The only final diagnosis of uterine adenomyosis is a histologic diagnosis based on pathology evaluation of the uterus after hysterectomy.

Only very experienced operators are able to diagnose adenomyosis by using Transvaginal Ultrasound (TVUS) or Magnetic Resonance Imaging (MRI). As these techniques require high experience and expertise it is difficult to apply them in routine diagnostics. Furthermore, the results of TVUS are operator-dependent (Dueholm M Bestt Practice & Research Clinical Obstetrics and Gynaecology 2006; 20(4):569-582) The detection of adenomyosis by transvaginal ultrasound requires also adequate ultrasound equipment and the result may depend from the specific ultrasound device used in the assessment and the subjective analysis of ultrasound images by a physician.

Among the different adenomyosis types, diffuse adenomyosis is even more difficult to detect by imaging techniques and requires an experienced sonographer. Also, access to imaging equipment is limited, especially among primary healthcare professionals, and requires trained staff and specialized resources

Therefore, a non-invasive blood-based test would allow medical assessment of adenomyosis without a need for imaging device, reduce the inter operator variability and enable a more standardized diagnosis of the condition. (Chapron C et al. Hum Reprod Update. 2020; 26:392-411).

CA125 has been reported in the differential diagnosis of uterine adenomyosis and myoma however the authors found only limited diagnostic accuracy (Kicheol Kil et al. Eur J Obstet Gynecol Reprod Biol. 2015 February; 185:131-5).

CA125 serum levels were found to be useful for predicting the prognosis of adenomyosis before and after interventional surgical therapy (Y. Mu et al. Int J Clin Exp Med 2015; 8(6): 9549-9554)

Secreted frizzled-related protein 4 (sFRP4) is a glycoprotein which belongs to a family of secreted proteins that act as antagonists of the Wnt ligands. It is also known by its synonyms FRP-4, frpHE (FRP Human Endometrium), FRPHE, FRZB-2 sFRPs encompass Wnt-binding domains and are soluble regulators of Wnt signaling pathways. sFRP4 inhibits the canonical Wnt signalling pathway, which normally induces cell proliferation and decrease apoptosis.

The SFRP4 gene is normally expressed in various tissues including endometrial stroma (higher expression in the proliferative phase of the menstrual cycle), pancreas, stomach, colon, lung, skeletal muscle, testis, ovary, kidney, heart, brain, mammary gland, cervix, eye, bone, prostate, and liver. The over expression of SFRP4 is associated with a variety of pathologies including bone, skin, renal, endocrinal and cancer (Pawar N et al. Secreted frizzled related protein 4 (sFRP4) update: A brief review Cellular Signalling 2018; 45:63-70; S. Pohl et al. Tumor Biol. 2015; 36:143-152).

In addition sFRP4 has been widely reported to be over expressed in Type 2 Diabetes Mellitus. (e.g. T. Mahdi et al. Cell Metabolism 16, 625-633, Nov. 7, 2012).

Estrogen and progesterone are found to regulate the expression of SFRP4 during the endometrial cycle (higher expression in the proliferative phase of the menstrual cycle). During ovulation, sFRP4 increases apoptosis to facilitate the process.

The role of sFRP4 in the development of endometrium and the up-regulation in ovarian and endometrial cancer as well as the up-regulation of sFRP4 by hCG in animals with endometriosis are described in a Baboon model: (Sherwin J R A et al. Endocrinology 2010; 151 (10): 4982-4993).

WO2001032920 describes a screening method for genes and gene products which are associated with endometriosis by comparing the pattern of gene expression in a diseased endometrium to the pattern of gene expression in a healthy endometrium. Among other gene products, the expression level of SFRP4 was found to be different compared to the healthy control group.

Furthermore, WO2007090872 describes antibodies against secreted frizzled related protein-4 (sFRP4) and their use for the detection of sFRP4.

Pawar et al. described sFRP4 as a protein marker to detect endometriosis in serum In a study comprising 21 patients and 22 healthy controls they reported an increase in sFRP4 serum levels for the group of endometriosis patients versus the control group and a significantly increase of sFRP4 levels in endometriosis grade 3 and 4 according to the revised American Society of Reproductive Meicine (rASRM). sFRP4 was quantified by an ELISA assay (Pawar N M et al. Indian Journal of Clinical Biochemistry 2016; 31(Supplement 1):S1-S129).

However, there is a high need for a non-invasive diagnosis of adenomyosis by using a biomarker, which allow a reliable and early assessment of patients exhibiting signs and symptoms of adenomyosis.

The present invention, therefore, provides means and methods complying with these needs.

### SUMMARY OF THE INVENTION

In a first aspect, the present invention relates to a method of assessing whether a patient has adenomyosis or is at risk of developing adenomyosis, comprising determining the amount or concentration of sFRP4 in a sample of the patient, and comparing the determined amount or concentration to a reference.

In a second aspect, the present invention relates to a method of selecting a patient for therapy of adenomyosis, particular a drug-based therapy, a pain management therapy or a surgical therapy comprising determining the amount or concentration of sFRP4 in a sample of the patient, and comparing the determined amount or concentration to a reference.

In a third aspect, the present invention relates to a method of monitoring a patient suffering from adenomyosis or being treated for adenomyosis, comprising determining the amount or concentration of sFRP4 in a sample of the patient, and comparing the determined amount or concentration to a reference.

In a fourth aspect, the present invention relates to computer-implemented method assessing whether a patient has adenomyosis or is at risk of developing adenomyosis, said method comprising, receiving at a processing unit a value for the level of sFRP4 in a sample from a patient, processing the value received in step (a) with the processing unit, wherein said processing comprises retrieving from a memory one or more threshold values for the level of sFRP4 and comparing the value received in step (a) with the one or more threshold values, and assessing whether a patient has adenomyosis or is at risk of developing adenomyosis via an output device, wherein said assessment is based on the results of step (b).

### LIST OF FIGURES

FIG. 1a: Box plot of sFRP4 in adenomyosis cases (N=124) and controls (N=177)

FIG. 1b: Receiver Operator Curve (ROC) analyses for biomarker sFRP4. The AUC value of the ROC analysis for adenomyosis versus controls without adenomyosis was 0.62 x-axis=specificity, y-axis=sensitivity

FIG. 2a: Box plot of CA125 in adenomyosis cases (N=124) and controls (N=177). The AUC value of the ROC analysis for adenomyosis versus controls without adenomyosis was 0.51. x-axis=specificity, y-axis=sensitivity

FIG. 2b: Receiver Operator Curve (ROC) analyses for biomarker CA125.

### DETAILED DESCRIPTION OF THE INVENTION

We show for the first time that sFRP4 measured in blood is increased in women with adenomyosis compared to controls. There is an unmet medical need for a non-invasive blood test for the diagnosis and assessment of adenomyosis.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “indicator” as used herein, refers to a sign or signal for a condition or is used to monitor a condition. Such a “condition” refers to the biological status of a cell, tissue or organ or to the health and/or disease status of an individual. An indicator may be the presence or absence of a molecule, including but not limited to peptide, protein, and nucleic acid, or may be a change in the expression level or pattern of such molecule in a cell, or tissue, organ or individual. An indicator may be a sign for the onset, development or presence of a disease in an individual or for the further progression of such disease. An indicator may also be a sign for the risk of developing a disease in an individual.

In the context of present invention, the term “biomarker” refers to a substance within a biological system that is used as an indicator of a biological state of said system. In the art, the term “biomarker” is sometimes also applied to means for the detection of said endogenous substances (e.g. antibodies, nucleic acid probes etc., imaging systems). In the context of present invention, the term “biomarker” shall be only applied for the substance, not for the detection means. Thus, biomarkers can be any kind of molecule present in a living organism, such as a nucleic acid (DNA, mRNA, miRNA, rRNA etc.), a protein (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA) or a substance that has been internalized by the organism or a metabolite of such a substance.

The term “sFRP4”, “Secreted frizzled-related protein 4” is also called FRP-4, FRPHE, sFRP-4, secreted frizzled related protein. The secreted frizzled-related proteins (sFRPs) comprise a family of five proteins in mammals encompassing Wnt-binding domains. They are soluble regulators of Wnt signaling pathways that were first identified as antagonists of the Wnt/B-catenin pathway during embyonic development (Kawano, Y and Krypta, R. Secreted antagonists of the Wnt signalling pathpay. J. Cell Sci. 116, 2627.2634, doi: 10.10242/jcs.00623 (2003).

sFRP4 inhibits the canonical Wnt signalling pathway, which normally induces cell proliferation and decrease apoptosis. Furthermore, high serum levels of sFRPs have been documented in pathological conditions such as obesity, diabetes and osteoporosis (Belaya, Z. E. et al; Osteoporos 2013; Int. 24: 2191-2199. The terms polypeptide, peptide and protein are used interchangeable throughout this specification.

The sFRP4 protein is comprised of 346 amino acids with a predicted molecular weight of 39.9 kDa and an actual molecular weight of approximately 50-55 kDa. The sFRP4 protein folds into two independent domains. The N-terminus contains a secretion signal peptide followed by a ˜120 amino acid cysteine-rich domain (CRD). The CRD is 30-50% identical to the extracellular putative Wnt-binding domain of frizzled (Fzd) receptors and is characterized by the presence of ten cysteine residues at conserved positions. These cysteines form a pattern of disulfide bridges.

sFRP4 as used herein encompasses also variants of the aforementioned specific sFRP4 polypeptides. Such variants have at least the same essential biological and immunological properties as the specific sFRP4 polypeptides. In particular, they share the same essential biological and immunological properties if they are detectable by the same specific assays referred to in this specification, e.g., by ELISA assays using polyclonal or monoclonal antibodies specifically recognizing the said sFRP4 polypeptides. A preferred assay is described in the accompanying Examples. Moreover, it is to be understood that a variant as referred to in accordance with the present invention shall have an amino acid sequence which differs due to at least one amino acid substitution, deletion and/or addition wherein the amino acid sequence of the variant is still, preferably, at least about 50%, at least about 60%, at least about 70%, at least about 80%, at least about 85%, at least about 90%, at least about 92%, at least about 95%, at least about 97%, at least about 98%, or at least about 99% identical with the amino sequence of the specific sFRP4 polypeptides, preferably with the amino acid sequence of human sFRP4, more preferably over the entire length of the specific sFRP4, e.g. of human sFRP4. The degree of identity between two amino acid sequences can be determined as described above. Variants referred to above may be allelic variants or any other species specific homologs, paralogs, or orthologs. Moreover, the variants referred to herein include fragments of the specific sFRP4 polypeptides or the aforementioned types of variants as long as these fragments have the essential immunological and biological properties as referred to above. Such fragments may be, e.g., degradation products of the sFRP4 polypeptides. Further included are variants which differ due to posttranslational modifications such as phosphorylation or myristylation.

“CA-125”, the Carbohydrate antigen 125, sometimes named as Cancer Antigen 125 or Tumor Antigen 125, is a mucin-type glycoprotein, produced by the MUC16 gene, and associated with the cellular membrane. CA-125 is a biomarker for epithelial cell ovarian cancer being derived from coelomic epithelia including the endometrium, fallopian tube, ovary, and peritoneum. Diagnostic use of CA-125 is limited to endometriosis stages III and IV (moderate and severe endometriosis) with moderate sensitivity.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to inflammatory diseases, infectious diseases, cutaneous conditions, endocrine diseases, intestinal diseases, neurological disorders, joint diseases, genetic disorders, autoimmune diseases, traumatic diseases, and various types of cancer.

The term Endometriosis is defined as a disease characterized by the presence of endometrium-like epithelium and/or stroma outside the endometrium and myometrium, usually with an associated inflammatory process (International working group of AAGL ESGE ESHRE and WES, et al.; ESHRE 2022 Guideline Endometriosis).

In contrast, the term Adenomyosis is defined as the presence of ectopic endometrial tissue (endometrial stroma and glands) within the myometrium (International working group of AAGL ESGE ESHRE and WES, et al., 2021). Adenomyosis is not considered a form or subtype of endometriosis and hence not covered in the guideline (ESHRE Guideline 2022, page 8).

Adenomyosis is further defined as ‘the benign invasion of endometrium into the myometrium (the muscular layer of the uterus) producing a diffusely enlarged uterus which microscopically exhibits ectopic, non-neoplastic endometrial glands and stroma surrounded by hypertrophic and hyperplastic myometrium’ (Bird C et al. Am J Obstet Gynecol 1972; 112: 583e593).

Different classifications of adenomyosis subtypes have been suggested. In 2012 Kishi et al classified adenomyosis based on Magnetic Resonance Imaging (MRI) findings, into 4 subtypes I-IV, according to whether it is located in the outer or the inner uterine layer (Y Kishi et al. Am J Obstet Gynecol. 2012; 207: 114.el-114.e7).

In 2018, Bazot et al suggested three distinct adenomyosis sybtypes, depending on the morphology, the extent of the myometrial invasion and location: internal adenomyosis, external adenomyosis and adenomyomas. Internal adenomyosis was further classified into focal, diffuse and superficial adenomyosis (Bazot M et al. Fertil Steril. 2018; 109:389-397). Women with focal adenomyosis present with lesions of a restricted area of hypertrophic and distorted endometrium and myometrium, which are usually embedded within the myometrium. Focal adenomyosis can be further subdivided into adenomyoma with more or less clear borders and mainly solid characteristics and cystic adenomyosis characterized mainly by the presence of a single adenomyotic cyst within the myometrium (also known as juvenile cystic adenomyosis in women younger than 30 years old). Diffuse adenomyosis as the extensive form of the disease is characterized by foci of endometrial mucosa (glands and stroma) scattered throughout the uterine musculature. Polypoid adenomyomas describes endometrial masses composed of predominantly endometrioid glands and a stromal component predominantly of smooth muscle. Adenomyomatous polyps in the uterine cervix represent rare forms of adenomyosis. These lesions are important because they must be differentiated from adenoma malignum (G Grimbizis et al. FERTILITY PRESERVATION 2014; 101(2):472). (19).

The term adenomyosis encompasses internal adenomyosis, external adenomyosis and adenomyoma. It also encompasses adenomyosis subtypes as diffuse, focal and superficial adenomyosis, adenomyotic cysts, adenomyoma, cystic adenomyosis, typical or atypical polypoid adenomyosis and adenomyomatous polyps, retroperitoneal adenomyomas, adenomyotic nodules (Bazot M et al. Fertil Steril. 2018; 109:389-397; G Grimbizis et al. FERTILITY PRESERVATION 2014; 101(2):472). Adenomyosis subtypes I-IV as classified based on Magnetic Resonance Imaging (MRI) findings.

Patients with adenomyosis can have a range of clinical presentations. The most common presentation of adenomyosis is abnormal menstrual bleeding (menorraghia), painful periods (dysmenorrhea) and an enlarged tender uterus. These symptoms are common to a number of other gynaecological disorders in women. The pain can be sharp, knifelike pelvic pain and there may-be severe cramping during menstruation. Pain can occur unpredictably and intermittently throughout the menstrual cycle or it can be continuous. However, there could be also different forms of pain and pain can also worsen over time and may change in character.

The enlarged uterus can put pressure on the bladder and rectum and can cause difficulties with urination. Nausea has also been reported by women with adenomyosis. Less commonly reported symptoms are dyspareunia and chronic, erratic or constant pelvic pain. Similar symptoms are reported in patients with endometriosis and there are women who are suffering from both diseases. Adenomyosis is also associated with infertility and diagnosed in about 20% of infertile women undergoing assisted reproductive technologies (ART) (S. Vannuccini et al. Fertility and Sterility® Vol. 109, No. 3, March 2018). Patients with adenomyosis often have other associated gynecologic conditions such as endometriosis or leiomyomas, therefore making the diagnosis and evaluating response to treatment challenging.

The prevalence of adenomyosis varies widely, with an average rate of 20%-25%. Approximately 20% of adenomyosis cases involve women of reproductive age (<40 years), with the remaining 80% occurring in women of late reproductive age (40-50 years). One-third of women affected by adenomyosis are asymptomatic (Pontis A et al. Gynecol Endocrinol, 2016; 32(9): 696-700).

Preferably, the term “therapy” as used in the context for a method of assessing whether a patient has adenomyosis or is at risk of developing adenomyosis encompasses the deterioration and worsening of symptoms in order to either prevent the disease deterioration altogether or significantly decrease its impact upon the patient, such as reducing lower abdominal pain in the patient and heavy bleeding during menstruation, reduction of uterus size and before fertility treatments to improve the chances of pregnancy in infertile women with adenomyosis

A drug-based therapy encompasses nonhormonal drugs (i.e. nonsteroidal anti-inflammatory drugs (NSAIDs), hormonal treatments (i.e., progestins as i.e. Norethindrone Acetate, oral contraceptives, combined oral contraceptives (COCS), gonadotropin-releasing hormone analogues ([GnRH] analogues), levonorgestrel-IUS (levonorgestrel-releasing intrauterine system), selective progesterone receptor modulators, aromatase inhibitors, valproic acid, anti-platelets therapy (S. Vannuccini et al. Fertility and Sterility® Vol. 109, No. 3, March 2018; A. Pontis et al. Gynecol Endocrinol. 2016; 3590: 1-5).

A surgical therapy encompasses the removal of the uterus (hysterectomy) and uterus preserving surgery as e.g. the surgical excision of adenomyotic lesions or cysts (adenomyomectomy, cystectomy) or Uterine Artery Embolization (UAE).

The term “VAS”, the Visual Analog Scale, is an instruments to assess the intensity of pain. The VAS consists of a 10-cm long horizontal line with its extremes marked as ‘no pain’ and ‘worst pain imaginable’. Each patient ticks her pain level on the line and the distance from ‘no pain’ on the extreme left to the tick mark is measured in centimeters, yielding a pain score from 0 to 10. ‘No pain’ corresponds to a pain score of 0, ‘worst pain imaginable’ corresponds to a pain score of 10. In women with adenomyosis dysmenorrhea is associated with the highest perception of pain with a mean VAS score of about 6 (Cozzolino et al. Rev Bras Ginecol Obstet 2019; 41(3): 170-175).

As used herein, a “patient” means any mammal, that may benefit from the diagnosis, prognosis or treatment described herein. In particular, a “patient” is selected from the group consisting of laboratory animals (e.g. mouse, rat, rabbit, or zebrafish), domestic animals (including e.g. guinea pig, rabbit, horse, donkey, cow, sheep, goat, pig, chicken, camel, cat, dog, turtle, tortoise, snake, lizard or goldfish), or primates including chimpanzees, bonobos, gorillas and human beings. It is particularly preferred that the “patient” is a human being.

In particular, a “patient” according to the present invention are women with symptoms such as abnormal uterus bleeding e.g. heavy menstrual bleeding, prolonged menstrual bleeding, bleeding between periods, dysmenorrhea, severe menstrual cramps, abdominal pressure and bloating. Women (with or without symptoms of adenomyosis) often have inability of getting pregnant and are often infertile women undergoing assisted reproductive technologies (ART).

Furthermore, the patient suffering from adenomyosis is a woman in the reproductive age usually 14-50 years old; in particular a woman of 30-50 years.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount, concentration or level. The sample is an in vitro sample, it will be analyzed in vitro and not transferred back into the body.

The term “amount” as used herein encompasses the absolute amount of a biomarker as referred to herein, the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts measured from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the biomarker in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer-assisted. Thus, the comparison may be carried out by a computing device. The value of the measured or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provides the desired assessment in a suitable output format.

The expression “comparing the amount or concentration determined to a reference” is merely used to further illustrate what is obvious to the skilled artisan anyway. A reference concentration is established in a control sample

The term “reference sample” or “control sample” as used herein, refers to a sample which is analysed in a substantially identical manner as the sample of interest and whose information is compared to that of the sample of interest. A reference sample thereby provides a standard allowing for the evaluation of the information obtained from the sample of interest. A control sample may be derived from a healthy or normal tissue, organ or individual, thereby providing a standard of a healthy status of a tissue, organ or individual. Differences between the status of the normal reference sample and the status of the sample of interest may be indicative of the risk of disease development or the presence or further progression of such disease or disorder. A control sample may be derived from an abnormal or diseased tissue, organ or individual thereby providing a standard of a diseased status of a tissue, organ or individual. Differences between the status of the abnormal reference sample and the status of the sample of interest may be indicative of a lowered risk of disease development or the absence or bettering of such disease or disorder. A reference sample may also be derived from the same tissue, organ, or individual as the sample of interest but has been taken at an earlier time point. Differences between the status of the earlier taken reference sample and the status of the sample of interest may be indicative of the progression of the disease, i.e. a bettering or worsening of the disease over time.

The control sample may be an internal or an external control sample. An internal control sample is used, i.e. the marker level(s) is(are) assessed in the test sample as well as in one or more other sample(s) taken from the same subject to determine if there are any changes in the level(s) of said marker(s). For an external control sample the presence or amount of a marker in a sample derived from the individual is compared to its presence or amount in an individual known to suffer from, or known to be at risk of, a given condition; or an individual known to be free of a given condition, i.e., “normal individual”.

It will be appreciated by the skilled artisan that such external control sample may be obtained from a single individual or may be obtained from a reference population that is age-matched and free of confounding diseases. Typically, samples from 100 well-characterized individuals from the appropriate reference population are used to establish a “reference value”. However, reference population may also be chosen to consist of 20, 30, 50, 200, 500 or 1000 individuals. Healthy individuals represent a preferred reference population for establishing a control value.

For example, a marker concentration in a patient sample can be compared to a concentration known to be associated with a specific course of a certain disease. Usually the sample's marker concentration is directly or indirectly correlated with a diagnosis and the marker concentration is e.g. used to determine whether an individual is at risk for a certain disease. Alternatively, the sample's marker concentration can e.g. be compared to a marker concentration known to be associated with a response to therapy in a certain disease, the diagnosis of a certain disease, the assessment of the severity of a certain disease, the guidance for selecting an appropriate drug to a certain disease, in judging the risk of disease progression, or in the follow-up of patients. Depending on the intended diagnostic use an appropriate control sample is chosen and a control or reference value for the marker established therein. As also clear to the skilled artisan, the absolute marker values established in a control sample will be dependent on the assay used.

The terms “lowered” or “decreased” level of an indicator refer to the level of such indicator in the sample being reduced in comparison to the reference or reference sample.

The terms “elevated” or “increased” level of an indicator refer to the level of such indicator in the sample being higher in comparison to the reference or reference sample. E.g. a protein that is detectable in higher amounts in a fluid sample of one individual suffering from a given disease than in the same fluid sample of individuals not suffering from said disease, has an elevated level.

The term “measurement”, “measuring” or “determining” preferably comprises a qualitative, a semi-quanitative or a quantitative measurement.

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, γ, δ, ε, and μ. The type of heavy chain present defines the class of antibody, i.e. these chains are found in IgA, IgD. IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CH1. CH2. CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCyl-Cy2-Cy3), whereas the human IgG light chain is composed of two immunoglobulin domains linked from N- to C-terminus in the order VL-CL, being either of the kappa or lambda type (VK-CK or VA.- CA.). Exemplified, the constant chain of human IgG comprises 447 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5^{th}ed. U.S. Department of Health and Human Service, National Institutes of Health, Bethesda, MD. The “EU index as in Kabat” refers to the residue numbering of the human IgG IEU antibody. Accordingly, CH domains in the context of IgG are as follows: “CH1” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The terms “full-length antibody”, “intact antibody”, and “whole antibody” are used herein interchangeably to refer to an antibody in its substantially intact form, not antibody fragments as defined below. The terms particularly refer to an antibody with heavy chains that contain an Fc region.

Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains, in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bispecific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

Accordingly, the term “antibody fragments” refers to a portion of an intact antibody, preferably comprising the antigen-binding region thereof. Antibody fragments include but are not limited to Fab, Fab′, F(ab′)₂, Fv fragments; diabodies; sdAb, nanobodies, scFv, di-scFvs, tandem scFvs, triabodies, diabodies, scDb, BiTEs, and DARTs.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

“Sandwich immunoassays” are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g. an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 37º C inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

An extremely versatile alternative sandwich assay format includes the use of a solid phase coated with the first partner of a binding pair, e.g. paramagnetic streptavidin-coated microparticles. Such microparticles are mixed and incubated with an analyte-specific binding agent bound to the second partner of the binding pair (e.g. a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay may include one or more washing step(s).

The term “detectably labeled” encompasses labels that can be directly or indirectly detected.

Directly detectable labels either provide a detectable signal or they interact with a second label to modify the detectable signal provided by the first or second label, e.g. to give FRET (fluorescence resonance energy transfer). Labels such as fluorescent dyes and luminescent (including chemiluminescent and electrochemiluminescent) dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling. In one embodiment detectably labeled refers to a label providing or inducible to provide a detectable signal, i.e. to a fluorescent label, to a luminescent label (e.g. a chemiluminescent label or an electrochemiluminescent label), a radioactive label or a metal-chelate based label, respectively.

Numerous labels (also referred to as dyes) are available which can be generally grouped into the following categories, all of them together and each of them representing embodiments according the present disclosure:

### (a) Fluorescent Dyes

Fluorescent dyes are e.g. described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oregon, USA) and Pierce Biotechnology, Inc. (Rockford, Ill.).

### (b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)3]2+(which releases a photon at ˜620 nm) regenerating with TPA (Tripropylamine) in liquid phase or liquid-solid interface are used as ECL-labels.

Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescense is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605, 5,591,581, 5,597,910, PCT published application WO90/05296, PCT published application WO92/14139, PCT published application WO90/05301, PCT published application WO96/24690, PCT published application US95/03190, PCT application US97/16942, PCT published application US96/06763, PCT published application WO95/08644, PCT published application WO96/06946, PCT published application WO96/33411, PCT published application WO87/06706, PCT published application WO96/39534, PCT published application WO96/41175, PCT published application WO96/40978, PCT/US97/03653 and U.S. patent application Ser. No. 08/437,348 (U.S. Pat. No. 5,679,519). Reference is also made to a 1994 review of the analytical applications of ECL by Knight, et al. (Analyst, 1994, 119: 879-890) and the references cited therein. In one embodiment the method according to the present description is practiced using an electrochemiluminescent label.

Recently also Iridium-based ECL-labels have been described (WO2012107419).

(c) Radioactive labels make use of radioisotopes (radionuclides), such as 3H, 11C, 14C, 18F, 32P, 35S, 64Cu, 68Gn, 86Y, 89Zr, 99TC, 111In, 1231, 1241, 1251, 131I, 133Xe, 177Lu, 211At, or 131Bi.

(d) Metal-chelate complexes suitable as labels for imaging and therapeutic purposes are well-known in the art (US 2010/0111861; U.S. Pat. Nos. 5,342,606; 5,428,155; 5,316,757; 5,480,990; 5,462,725; 5,428,139; 5,385,893; 5,739,294; 5,750,660; 5,834,461; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

### Embodiments

In a first aspect, the present invention relates to a method of assessing whether a patient has adenomyosis or is at risk of developing adenomyosis, comprising
a) determining the amount of sFRP4 in a sample of the patient, and
b) comparing the determined amount to a reference.

In embodiments, an elevated amount of sFRP4 in the sample of the patient is indicative of the presence or the risk of developing adenomyosis in the patient. In particular, an amount of sFRP4 in the sample of the patient is indicative of the presence or the risk of developing of adenomyosis in the patient if the amount of sFRP4 in the sample of the patient is higher than the amount of sFRP4 in a reference or reference sample. In particular, sFRP4 is detectable in higher amounts in a fluid sample of the patient assessed for the presence or risk of developing adenomyosis than in the same fluid sample of individuals not suffering or being at risk of developing adenomyosis.

In particular, an amount of sFRP4 elevated by 50% or more, is indicative of the presence or the risk of developing of adenomyosis. In particular, an amount of sFRP4 elevated by 100% or more, is indicative of the presence or the risk of developing of adenomyosis. In particular, an amount of sFRP4 elevated by 150% or more, is indicative of the presence or the risk of developing of adenomyosis. In particular, an amount of sFRP4 elevated by 200% or more, is indicative of the presence or the risk of developing of adenomyosis.

In embodiments, the sample of the patient is body fluid sample. In particular embodiments, the sample is a whole blood, serum or plasma sample. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient. In particular embodiments, the patient is a female human patient.

In particular, the assessment is performed without performing surgery. In particular the assessment is performed without assessing the presence or severity of adenomyosis in the patient using surgery.

In embodiment, the method of the present invention is an in vitro method.

In embodiments, the amount of sFRP4 is determined using antibodies, in particular using monoclonal antibodies. In embodiments, step a) of determining the amount of sFRP4 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmuno-assay (RIA), or immuno assays based on detection of luminescence, fluorescence, chemiluminescence or electrochemiluminescence.

In particular embodiments, step a) of determining the amount of sFRP4 in a sample of the patient comprises the steps of
i) incubating the sample of the patient with one or more antibodies specifically binding to sFRP4, thereby generating a complex between the antibody and sFRP4, and
ii) quantifying the complex formed in step i), thereby quantifying the amount of sFRP4 in the sample of the patient.

In particular embodiments, in step i) the sample is incubated with two antibodies, specifically binding to sFRP4. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-sFRP4 antibody/sFRP4/second anti-sFRP4 antibody complex. As the skilled artisan will readily appreciate it is nothing but routine experimentation to establish the time and conditions that are appropriate or that are sufficient for the formation of a complex either between the specific anti-sFRP4 antibody and the sFRP4 antigen/analyte (=anti-sFRP4 complex) or the formation of the secondary, or sandwich complex comprising the first antibody to sFRP4, sFRP4 (the analyte) and the second anti-sFRP4 antibody (=anti-sFRP4 antibody/sFRP4/second anti-sFRP4 antibody complex).

The detection of the anti-sFRP4 antibody/sFRP4 complex can be performed by any appropriate means. The detection of the first anti-sFRP4 antibody/sFRP4/second anti-sFRP4 antibody complex can be performed by any appropriate means. The person skilled in the art is absolutely familiar with such means/methods.

In certain embodiments a sandwich will be formed comprising a first antibody to sFRP4, sFRP4 (analyte) and the second antibody to SFRP4, wherein the second antibody is detectably labeled.

In one embodiment a sandwich will be formed comprising a first antibody to sFRP4, the sFRP4 (analyte) and the second antibody to SFRP4, wherein the second antibody is detectably labeled and wherein the first anti-SFRP4 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In embodiments, the method further comprising the assessment of the presence of dysmenorrhea and/or lower abdominal pain in the patient. In embodiments the presence of dysmenorrhea and/or lower abdominal pain is assessed according to the VAS scale. In embodiments, dysmenorrhea VAS score of 4 or higher indicated moderate or severe dysmenorrhea.

In a second aspect, the present invention relates to a method of selecting a patient for a therapy of adenomyosis, comprising
determining the amount or concentration of sFRP4 in a sample of the patient, and
comparing the determined amount or concentration to a reference.

In a further embodiment of the present invention, the therapy is in particular a drug-based therapy, a pain management therapy or a surgical therapy.

In embodiments, a patient is selected for therapy of adenomyosis if an elevated amount of sFRP4 in the sample of the patient is determined. In particular, a patient is selected for therapy of adenomyosis if the amount of sFRP4 in the sample of the patient is higher than the amount of sFRP4 in a reference or reference sample. In particular, a patient is selected for therapy of adenomyosis if the amount of SFRP4 is higher in a fluid sample of the patient than in the same fluid sample of individuals not suffering or being at risk of developing adenomyosis or not being selected for therapy of adenomyosis.

In particular, a patient is selected for therapy of adenomyosis if the amount of sFRP4 is elevated by 50% or more. In particular, a patient is selected for therapy of adenomyosis if the amount of sFRP4 is elevated by 100% or more. In particular, a patient is selected for therapy of adenomyosis if the amount of sFRP4 is elevated by 150% or more. In particular, a patient is selected for therapy of adenomyosis if the amount of sFRP4 is elevated by 200% or more.

In embodiments, the patient is selected for a therapy of adenomyosis selected from the group consisting of drug-based therapy or surgical therapy.

In embodiments, drug-based therapy of adenomyosis is inhibiting or targeting neurogenic inflammation and/or pain medication and/or hormonal therapy (e.g. hormonal contraceptives or ([GnRH] analogues).

In embodiments, a surgical therapy of adenomyosis encompasses among others hysterectomy, the removal of the uterus; uterus preserving surgery as e.g. the surgical excision of adenomyotic lesions; cysts (adenomyomectomy, cystectomy); Uterine Artery Embolization (UAE) or nerve-sparing surgery.

In embodiments, the sample of the patient is body fluid sample. In particular embodiments, the sample is a whole blood, serum or plasma sample. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient. In particular embodiments, the patient is a female human patient.

In embodiment, the method of the present invention is an in vitro method.

In embodiments, the amount of sFRP4 is determined using antibodies, in particular using monoclonal antibodies. In embodiments, step a) of determining the amount of sFRP4 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In particular embodiments, step a) of determining the amount of sFRP4 in a sample of the patient comprises the steps of
i) incubating the sample of the patient with one or more antibodies specifically binding to sFRP4, thereby generating a complex between the antibody and sFRP4, and
ii) quantifying the complex formed in step i), thereby quantifying the amount of sFRP4 in the sample of the patient.

In particular embodiments, in step i) the sample is incubated with two antibodies, specifically binding to sFRP4. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-sFRP4 antibody/sFRP4/second anti-sFRP4 antibody complex. As the skilled artisan will readily appreciate it is nothing but routine experimentation to establish the time and conditions that are appropriate or that are sufficient for the formation of a complex either between the specific anti-sFRP4 antibody and the sFRP4 antigen/analyte (=anti-sFRP4 complex) or the formation of the secondary, or sandwich complex comprising the first antibody to sFRP4, sFRP4 (the analyte) and the second anti-sFRP4 antibody (=anti-sFRP4 antibody/sFRP4/second anti-sFRP4 antibody complex).

The detection of the anti-sFRP4 antibody/sFRP4 complex can be performed by any appropriate means. The detection of the first anti-sFRP4 antibody/sFRP4/second anti-sFRP4 antibody complex can be performed by any appropriate means. The person skilled in the art is absolutely familiar with such means/methods.

In certain embodiments, a sandwich will be formed comprising a first antibody to sFRP4, sFRP4 (analyte) and the second antibody to sFRP4, wherein the second antibody is detectably labeled.

In one embodiment a sandwich will be formed comprising a first antibody to sFRP4, the sFRP4 (analyte) and the second antibody to sFRP4, wherein the second antibody is detectably labeled and wherein the first anti-sFRP4 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In embodiments, the method further comprising the assessment of the presence of dysmenorrhea and/or lower abdominal pain in the patient. In embodiments the presence of dysmenorrhea and/or lower abdominal pain is assessed according to the VAS scale. In embodiments, dysmenorrhea VAS score of 4 or higher indicated moderate or severe dysmenorrhea. In embodiments, scores of 3 or less indicate no or mild dysmenorrhea.

In embodiments, the method comprising calculating a ratio of the amount or concentration of sFRP4 and dysmenorrhea, of the amount or concentration of sFRP4 and lower abdominal pain according to the VAS scale, or the amount or concentration of sFRP4 and the amount or concentration of CA-125.

In a third aspect, the present invention relates to a method of monitoring a patient suffering from adenomyosis or being treated for adenomyosis, comprising
a) determining the amount or concentration of sFRP4 in a sample of the patient, and
b) comparing the determined amount or concentration to a reference.

In embodiments, a patient suffering from adenomyosis is monitored to determine if the amount or concentration of sFRP4 is changing over time in a sample of the patient. In particular, a patient suffering from adenomyosis is monitored to determine if the amount or concentration of sFRP4 is increasing, decreasing or not changing over time. In embodiments, a patient suffering from adenomyosis is monitored if an elevated amount of sFRP4 in the sample of the patient is determined.

In embodiments, a patient being treated for adenomyosis is monitored to determine if the amount or concentration of sFRP4 is changing in a sample of the patient. In particular, a patient being treated for adenomyosis is monitored to determine if the amount or concentration of sFRP4 is increasing, decreasing or not changing. In particular, a patient being treated for adenomyosis is monitored to determine if the amount or concentration of sFRP4 is increasing, decreasing or not changing due to the therapy applied. In embodiments, a decreasing amount or concentration of sFRP4 in a patient being treated for adenomyosis is indicative of the therapy being effective. In embodiments, an unaltered or increasing amount or concentration of sFRP4 in a sample of the patient being treated for adenomyosis is indicative of the therapy being ineffective, i.e. an unaltered or increasing amount or concentration of sFRP4 in a sample of the patient being treated for adenomyosis is indicative of persisting or recurring adenomyosis.

In particular embodiments, therapy is adapted if an unaltered or increasing amount or concentration of sFRP4 in a sample of the patient being treated for adenomyosis is determined.

In embodiments, the patient is monitored several times at different time points. In embodiments, the patient is monitored several times within a time frame of weeks, months or years. In particular embodiments, a patient is monitored is once a months or once a year. In embodiments, a patient suffering from adenomyosis is monitored once a months or once a year after diagnosis of adenomyosis. In embodiments, a patient being treated for adenomyosis is monitored once after therapy, in particular once after surgical therapy. In particular, the patient being treated for adenomyosis is monitored once a months or once a year to determine the efficacy of treatment and/or the recurrence of adenomyosis.

In embodiments, therapy of adenomyosis is selected from the group consisting of drug-based therapy or surgical therapy. In embodiments drug-based therapy of adenomyosis is inhibiting or targeting neurogenic inflammation and/or pain medication and/or hormonal therapy (e.g. hormonal contraceptives or ([GnRH] analogues). In embodiments a surgical therapy of adenomyosis encompasses among others hysterectomy, the removal of the uterus, uterus preserving surgery as e.g. the surgical excision of adenomyotic lesions, cysts (adenomyomectomy, cystectomy) or Uterine Artery Embolization (UAE) or nerve-sparing surgery.

In embodiments, the sample of the patient is a body fluid sample. In particular embodiments, the sample is a whole blood, serum or plasma sample. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient. In particular embodiments, the patient is a female human patient.

In embodiment, the method of the present invention is an in vitro method.

In embodiments, the amount of sFRP4 is determined using antibodies, in particular using monoclonal antibodies. In embodiments, step a) of determining the amount of sFRP4 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In particular embodiments, step a) of determining the amount of sFRP4 in a sample of the patient comprises the steps of
i) incubating the sample of the patient with one or more antibodies specifically binding to sFRP4, thereby generating a complex between the antibody and sFRP4, and
ii) quantifying the complex formed in step i), thereby quantifying the amount of sFRP4 in the sample of the patient.

In particular embodiments, in step i) the sample is incubated with two antibodies, specifically binding to sFRP4. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-sFRP4 antibody/sFRP4/second anti-sFRP4 antibody complex. As the skilled artisan will readily appreciate it is nothing but routine experimentation to establish the time and conditions that are appropriate or that are sufficient for the formation of a complex either between the specific anti-sFRP4 antibody and the sFRP4 antigen/analyte (=anti-sFRP4 complex) or the formation of the secondary, or sandwich complex comprising the first antibody to SFRP4, SFRP4 (the analyte) and the second anti-sFRP4 antibody (=anti-sFRP4 antibody/sFRP4/second anti-sFRP4 antibody complex).

The detection of the anti-sFRP4 antibody/sFRP4 complex can be performed by any appropriate means. The detection of the first anti-sFRP4 antibody/sFRP4/second anti-sFRP4 antibody complex can be performed by any appropriate means. The person skilled in the art is absolutely familiar with such means/methods.

In certain embodiments, a sandwich will be formed comprising a first antibody to sFRP4, sFRP4 (analyte) and the second antibody to sFRP4, wherein the second antibody is detectably labeled.

In one embodiment a sandwich will be formed comprising a first antibody to sFRP4, the sFRP4 (analyte) and the second antibody to sFRP4, wherein the second antibody is detectably labeled and wherein the first anti-sFRP4 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemi-luminescent dye.

In a fourth aspect, the present invention relates to a method for a computer-implemented method for assessing whether a patient has adenomyosis or is at risk of developing adenomyosis a patient, said method comprising
a) receiving at a processing unit a value for the level of sFRP4 in a sample from a patient,
b) processing the value received in step (a) with the processing unit, wherein said processing comprises retrieving from a memory one or more threshold values for the level of sFRP4 and comparing the value received in step (a) with the one or more threshold values, and
c) assessing whether a patient has adenomyosis or is at risk of developing adenomyosis a patient via an output device, wherein said assessment is based on the results of step (b).

The above-mentioned method is a computer-implemented method. Preferably, all steps of the computer-implemented method are performed by one or more processing units of a computer (or computer network). Thus, the assessment in step (c) is carried out by a processing unit. Preferably, said assessment is based on the results of step (b).

The value or values received in step (a) shall be derived from the determination of the level of the biomarker from a patient suffering adenomyosis as described elsewhere herein. Preferably, the value is a value for the concentration of the biomarker. The value will be typically received by the processing unit by uploading or sending the value to the processing unit. Alternatively, the value can be received by the processing unit by inputting the value via an user interface.

In an embodiment of the aforementioned method, the reference (or references) set forth in step (b) is (are) established from a memory. Preferably, a value for the reference is established from the memory.

In an embodiment of the aforementioned computer-implemented method of the present invention, the result of the assessment made in step c) is provided via a display, configured for presenting result.

In an embodiment of the aforementioned computer-implemented method of the present invention, the method may comprise the further step of transferring the information on the assessment made in step c) to the patient suffering from adenomyosis by using electronic medical records.

The following examples and figures are provided to aid the understanding of the present invention, the true scope of which is set forth in the appended claims. It is understood that modifications can be made in the procedures set forth without departing from the spirit of the invention

### EXAMPLES

### Example 1: Diagnostic Performance of Biomarker sFRP4 and Biomarker CA-125 in Women with Adenomyosis and Controls in Samples from a Multicenter Study

The case group is comprised of patients diagnosed with adenomyosis by ultrasound or by laparoscopic visualization. The control group includes women without adenomyosis and without endometriosis. Inclusion criteria for the case and the control group were the presence of pelvic pain/infertility, scheduled for laparoscopy or laparoctomy and age between 18-45 years. Exclusion criteria for the case group were pregnancy,/breast feeding, malignancy, recurrent adenomyosis and endometriosis and laparoscopy/laparotomy for another reason≤6 months.

sFRP4 was measured with a pre-commercial ECLIA assay for sFRP4, a sandwich-immunoassay which was developed for the cobas Elecsys® ECLIA platform (ECLIA Assay from Roche Diagnostics, Germany). The assay comprises a biotinylated and a ruthenylated monoclonal antibody that specifically binds sFRP4. 49 μL were used from each serum sample and measured undiluted on a cobas e 601 analyzer (Roche Diagnostics, Germany). The Elecsys® Electro-Chemiluminescence (ECL) technology and assay method is briefly described below for the determination of CA 125 II.

The concentration of CA-125 was determined by a cobas e 601 analyzer. Detection of CA 125 II with a cobas e 601 analyzer is based on the Elecsys® Electro-ChemiLuminescence (ECL) technology. In brief, biotin-labelled and ruthenium-labelled antibodies are combined with the respective amount of undiluted sample and incubated on the analyzer. Subsequently, streptavidin-coated magnetic microparticles are added and incubated on the instrument in order to facilitate binding of the biotin-labelled immunological complexes. After this incubation step the reaction mixture is transferred into the measuring cell where the beads are magnetically captured on the surface of an electrode. ProCell M Buffer containing tripropylamine (TPA) for the subsequent ECL reaction is then introduced into the measuring cell in order to separate bound immunoassay complexes from the free remaining particles. Induction of voltage between the working and the counter electrode then initiates the reaction leading to emission of photons by the ruthenium complexes as well as TPA. The resulting electrochemiluminescent signal is recorded by a photomultiplier and converted into numeric values indicating concentration level of the respective analyte.

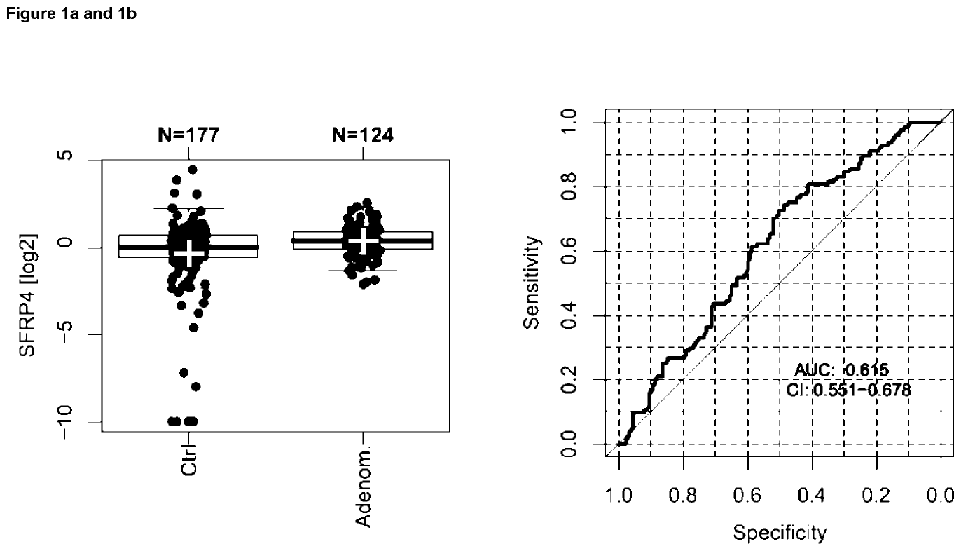

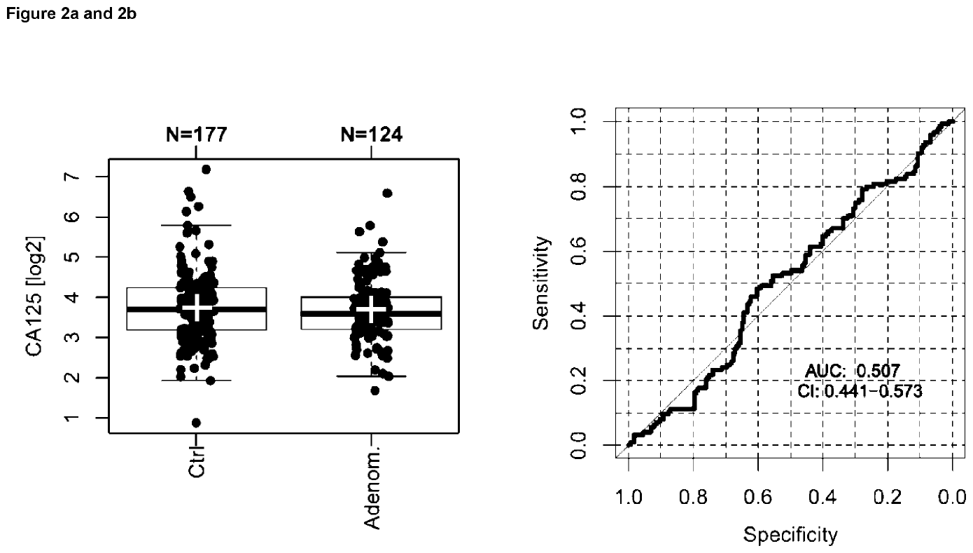

Receiver Operating Characteristic (ROC) curves were generated (see FIG. 1 for sFRP4 and FIG. 2 for Ca-125). The model performance is determined by looking at the area under the curve (AUC). The best possible AUC is 1 while the lowest possible is 0.5. Optimal cut-offs were selected using Youden's index (maximized sum of sensitivity plus specificity-1).

TABLE 1
Diagnostic performance of biomarkers sFRP4 and
CA-125 in adenomyosis cases and controls.

| | | Wilcoxon | N | N |
|---|---|---|---|---|
| Biomarker | AUC | p-value | (cases) | (controls) |
| sFRP4 | 0.615 | 0.001 | 124 | 177 |
| CA-125 | 0.507 | 0.829 | 124 | 177 |

In this group the diagnostic performance of sFRP4 to distinguish adenomyosis cases versus controls is higher compared to the diagnostic performance of the biomarker CA-125.

1. A method of assessing whether a patient has adenomyosis or is at risk of developing adenomyosis and/or selecting a patient for a therapy of adenomyosis, the method comprising
determining the amount or concentration of sFRP4 in a sample of the patient, and
comparing the determined amount or concentration to a reference.

2. (canceled)

3. The method of claim 1, wherein a therapy of adenomyosis is in selected from a drug-based therapy, a pain management therapy, and/or a surgical therapy.

4. A method of monitoring a patient suffering from adenomyosis or being treated for adenomyosis, the method comprising
determining the amount or concentration of sFRP4 in a sample of the patient, and
comparing the determined amount or concentration to a reference.

5. The method of claim 1, wherein an elevated amount or concentration of sFRP4 in the sample of the patient is indicative of the presence of adenomyosis in the patient.

6. The method of claim 1, wherein the sample is body fluid.

7. The method of claim 1, wherein the sample is blood, serum or plasma.

8. The method of claim 1, wherein the subject is a female patient, in particular a human female patient.

9. A computer-implemented method assessing whether a patient has adenomyosis or is at risk of developing adenomyosis, said method comprising
a) receiving at a processing unit a value for the level of sFRP4 in a sample from a patient;
b) processing the value received in step (a) with the processing unit, wherein said processing comprises retrieving from a memory one or more threshold values for the level of sFRP4 and comparing the value received in step (a) with the one or more threshold values; and
c) assessing whether a patient has adenomyosis or is at risk of developing adenomyosis via an output device, wherein said assessment is based on the results of step (b).

10. The method of claim 8, wherein the female patient is a human female patient.

11. The method of claim 1, wherein determining the amount of sFRP4 in a sample of the patient comprises performing an immunoassay.

12. The method of claim 1, wherein determining the amount of sFRP4 in a sample of the patient comprises
incubating the sample of the patient with one or more antibodies specifically binding to sFRP4, thereby generating a complex between the antibody and sFRP4, and
quantifying the complex, thereby quantifying the amount of sFRP4 in the sample of the patient.

13. The method of claim 12, wherein the sample is incubated with two antibodies specifically binding to sFRP4.

14. The method of claim 13, wherein a sandwich will be formed comprising a first antibody to sFRP4, the sFRP4 (analyte) and a second antibody to sFRP4, wherein the second antibody is detectably labeled.

15. The method of claim 14, wherein the first sFRP4 antibody is capable of binding to a solid phase or is bound to a solid phase.

16. The method of claim 14, wherein the second antibody is detectably labeled with a luminescent dye.

17. The method of claim 16, wherein the luminescent dye is a chemiluminescent dye or an electrochemiluminescent dye.

18. The method of claim 12, wherein the sample is incubated with a biotinylated first monoclonal sFRP4-specific antibody (or fragment thereof) and a ruthenylated second monoclonal sFRP4-specific antibody (or fragment thereof).

19. The method of claim 1, further comprising the assessment of the presence of dysmenorrhea and/or lower abdominal pain in the patient.

20. The method of claim 19, wherein the presence of dysmenorrhea and/or lower abdominal pain is assessed according to the VAS scale.

21. The method of claim 11, wherein the immunoassay is selected from the group consisting of enzyme linked immunosorbent assay (ELISA), enzyme immunoassay (EIA), radioimmuno assay (RIA), and immuno assays based on detection of luminescence, fluorescence, chemiluminescence or electrochemiluminescence.
